(19) United States
(12) Patent Application Publication
(10) Pub. No.: US 2013/0253447 A1
(43) Pub. Date: Sep. 26, 2013

(54) NEEDLE PLACEMENT SYSTEM

(71) Applicants: Brian W. Ball, Maple Grove, MN (US); Keith A. Miesel, St. Paul, MN (US); Mary M. Morris, Shoreview, MN (US); Luis E. Fesser, Woodbury, MN (US); Jonathan P. Bogott, Crystal, MN (US)

(72) Inventors: Brian W. Ball, Maple Grove, MN (US); Keith A. Miesel, St. Paul, MN (US); Mary M. Morris, Shoreview, MN (US); Luis E. Fesser, Woodbury, MN (US); Jonathan P. Bogott, Crystal, MN (US)

(73) Assignee: Medtronic, Inc.

(21) Appl. No.: 13/426,045

(22) Filed: Mar. 21, 2012

(51) Int. Cl. A61M 5/00

(52) U.S. Cl. 604/272; 604/288.01

(57) ABSTRACT

A needle and needle system that aids in determining needle position with respect to an implanted device. The system determines needle position by detecting changes in electrical characteristics and the system further may generate a cue to indicate proper needle placement within an implanted device. Methods for detecting needle position with respect to an implanted infusion device are also disclosed.

### TECHNICAL FIELD

This invention relates to systems, devices and methods for sensing and monitoring needle placement into an implanted medical device.

### BACKGROUND

Various types of implanted medical devices, such as implanted drug pumps, are used to deliver controlled volumes of a therapeutic fluid substance (e.g. a drug) within a patient's body. These pumps generally have reservoirs that may be accessed through ports, which may be self-sealing and may provide a drug suspension or solution from the device.

After the medical device is implanted within a patient, it may be desirable to fill, refill, flush out, or change fluid in a reservoir or other portion of the device. Typically, this is accomplished by a health care provider (HCP), for example a clinician. The HCP typically locates the device access port by palpitating a patient's skin, as the access port typically protrudes from the infusion pump. The HCP then inserts a needle or similar device advancing through the patient's skin into the implanted device to dispense or remove the intended therapeutic fluid substance.

Because the implanted medical device cannot be directly viewed, care must be taken to ensure proper needle placement into the device before injecting a therapeutic substance. If the needle misses the device, the therapeutic substance may be dispensed in the patient's body resulting in delivery of an improper amount and at an improper location, with potentially adverse consequences for the patient.

### SUMMARY OF THE INVENTION

The present invention provides, in one aspect, a needle detection system for determining needle position with respect to an implanted infusion device comprising:
a hollow needle having a sharp distal end;
a needle electrode located at a first distance along the needle from the distal end;
a second electrode; and
a detector electrically coupled to the first and second electrode, the detector providing needle insertion position information when the needle is inserted in an implanted medical device.

The invention provides, in another aspect, a position-indicating needle comprising:
a hollow needle having a sharp distal end;
a needle electrode located at a first distance along the needle from a distal end; and
a second electrode.

Still other aspects of the invention provide a method for detecting needle position with respect to an implanted infusion device, the method comprising:
sensing changes in electrical characteristics as a needle electrode enters into an
implanted infusion device; and
determining whether the sensed changes in electrical characteristics are indicative of needle entry into the implanted infusion device.

Such needle, needle system and method have particular use when refilling, flushing or changing fluid in an implanted infusion device.

### BRIEF DESCRIPTION OF THE DRAWING

FIG. 1 is a schematic illustration of an infusion system implanted in a patient.

FIG. 2 is a block diagram depicting components of an implanted infusion system of FIG. 1.

FIG. 3 is a cross-sectional view of a portion of the implanted infusion device of FIG. 1.

FIG. 4 is a side view of a needle embodiment with a needle placement verification system.

FIG. 5A is a side view, partially in section of another needle embodiment.

FIG. 5B is a side view, partially in section of yet another needle embodiment.

FIG. 5C is a perspective view of another needle embodiment.

FIG. 5D shows a top, side and bottom view of FIG. 5C.

FIGS. 6A-D are schematic illustrations of various needle insertion stages into an implanted infusion device.

FIG. 7 is a schematic illustration of a needle integrity or calibration system.

FIG. 8 shows a typical signal response to needle entry into a septum of an implanted infusion device.

FIG. 9 is a flow diagram showing needle insertion detection in an implanted device in accordance with the teachings described herein.

The drawings are not to scale. Like numbers used in the figures refer to like components, steps and the like. However, the use of such numbers to label a component in a given figure is not intended to limit a component in another figure labeled with the same number.

### DETAILED DESCRIPTION

The present disclosure describes systems, devices and methods that can be used to detect needle entry into an implanted infusion device. Such needle detection may be accomplished by monitoring changes in electrical characteristics such as changes in electrical resistance, impedance, current, voltage, frequency, or signal strength when a needle enters an implanted infusion device.

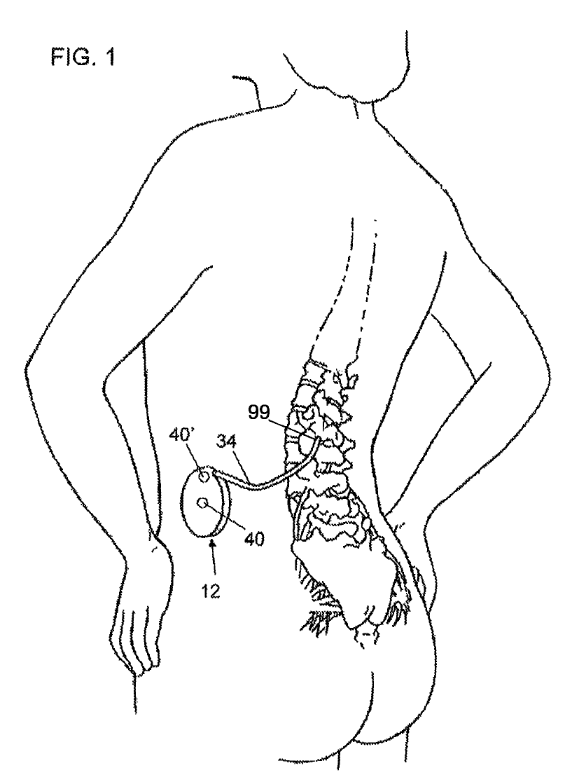

FIG. 1 shows an implanted infusion device 12 having two port assemblies 40, 40′ implanted in a patient. Infusion device 12 may include one, two, three, or any number of port assemblies. As shown in FIG. 1, a catheter 34 is connected to infusion device 12. Distal portion 99 of catheter 34, which may have one or more openings through which fluid may flow, is positioned at or near a target location of a patient to deliver fluid from infusion device 12 to the target location. The target location depicted in FIG. 1 is the patient's intrathecal space surrounding the spinal canal. It will be understood, however, that any region of a patient's body may serve as a target location depending on the conditions, disease, or disorder to be treated. Port assemblies 40, 40′ can be accessed percutaneously by a needle (not shown in FIG. 1), through which fluid may be delivered to infusion device 12.

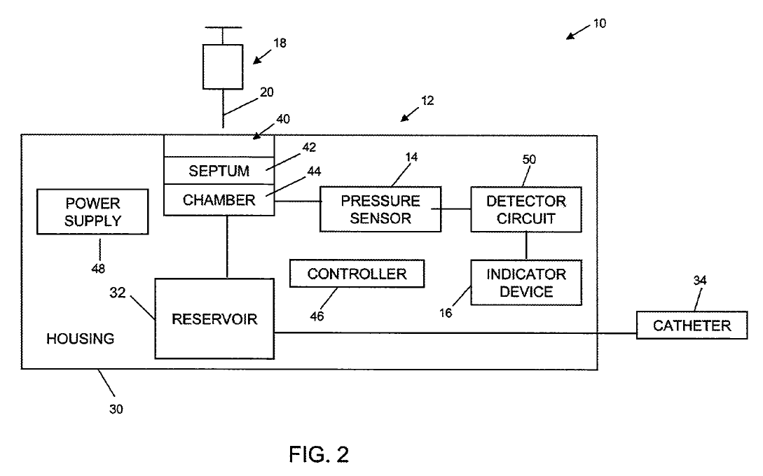

Infusion device 12 may be any device capable of delivering fluid to a patient. For example, infusion device 12 may be an access port, e.g. a vascular access port, through which a solution or therapeutic substance from a needle may be delivered through a catheter to a patient, or may be a device having a reservoir (shown in FIG. 2) for holding solutions containing a therapeutic substance to be delivered over a period of time, such as devices with fixed or variable rate pumps, programmable pumps, or the like. Infusion devices having a reservoir will generally include a port assembly to allow for filling the reservoir.

Port assemblies 40, 40′, shown in FIG. 1, may for example respectively be a catheter access port and a fill port. As described in further detail below, fill port assembly 40 provides access to a reservoir 32 that retains a therapeutic substance. Exemplary devices having a catheter access port and a fill port include Medtronic's SYNCHROMED™ implanted infusion device, DePuy's CODMAN™ 3000 and OMT's LENUS PRO™ or other such implantable medical devices. Other exemplary implantable I.V. infusion port devices include Smiths Medical's PORT-A-CATHT™ and P.A.S PORT™, and Bard Medical's POWERPORT™. Any currently known or future developed implanted infusion device can also be used.

In some embodiments, multiple catheters may be coupled to infusion device 12 to target the same or different tissue sites within a patient. Thus, although a single catheter 34 is shown in FIG. 1, in other embodiments, infusion device 12 may include multiple catheters or catheter 34 may define multiple lumens for delivering different therapeutic substances or for delivering a therapeutic substance to different tissue sites within patient. Accordingly, in some embodiments, infusion device 12 may include a plurality of reservoirs for storing more than one type of therapeutic substance, with each such reservoir typically having its own access port. For ease of description, an infusion device 12 including a single reservoir is primarily discussed herein.

FIG. 2 shows a block diagram depicting systems and components in a representative system 10 that includes an implanted infusion device 12. Implanted infusion device 12 further includes a refill port 40, septum 42, chamber 44, reservoir 32, a pressure sensor 14, a detector 39, an indicator 16 and a power supply 48. Also depicted in FIG. 2 is a syringe assembly 18 including a needle 20 useful for percutaneously interfacing with the implanted infusion device 12. In general, infusion device 12 shown in FIG. 2 includes a housing 30 that typically will surround reservoir 32. Reservoir 32 may contain a therapeutic substance to be delivered to the patient, for example, via a catheter 34.

The therapeutic substance can be any infusion agent, product, or substance intended to have a therapeutic effect such as pharmaceutical compositions, genetic materials, biologics, and others (e.g., insulin, saline solution, fluoroscopy agents, antibiotics or the like). A pump, metering device, flow regulator or combination thereof can be provided for dictating the therapeutic substance flow from reservoir 32 in a desired fashion. The pump/metering device can assume a variety of forms, and device 12 can further include a propellant chamber associated with reservoir 32 for exerting a constant, positive pressure onto the contained therapeutic substance to ensure delivery to the outlet catheter 34. In other embodiments, the pump/metering device can be eliminated, especially where gravity, osmotic pressure or other driving forces may be used to deliver the therapeutic substance to the patient.

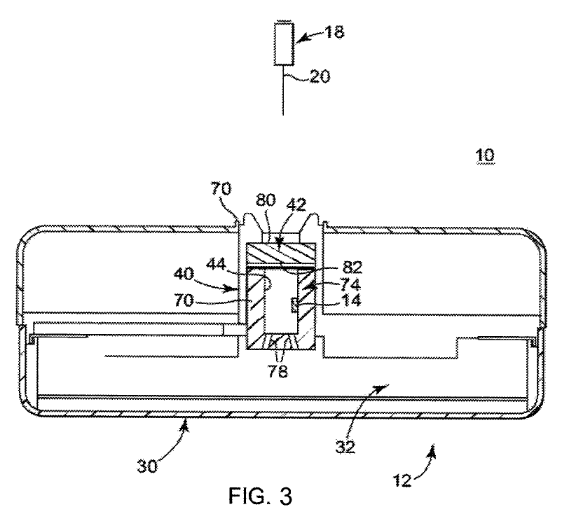

FIG. 3 is a simplified, cross-sectional view of a portion of system 10 and infusion device 12, housing 30, reservoir 32, and port assembly 40. In general, port assembly 40 is formed in an opening 70 of housing 30 such that port assembly 40 is exteriorly accessible relative to housing 30. Septum 42 may be disposed across port chamber 44 (referenced generally) defined by a wall of port assembly 40, such that septum 42 seals the opening 70 relative to the port chamber 44/reservoir 32.

Septum 42 can be made from any suitable sealing material or materials and may be electrically conducting or non-conducting. Typically, septum 42 may be made of an elastomeric material, for example, silicone rubber that is electrically non-conducting, able to be pierced or otherwise penetrated by a needle 20 and compatible with the therapeutic substance to be contained within reservoir 32. In various embodiments, port assembly 40 further includes a septum plug 74 used to retain septum 42 while providing a fluid-tight seal. Septum plug 74 defines the port chamber 44 to include drain holes 78 that allow fluids delivered to port chamber 44 to pass into reservoir 32. In some embodiments, a valve can be provided to further control liquid flow from port chamber 44 to reservoir 32. As a point of reference, relative to an arrangement of port assembly 40, septum 42 defines a first or exterior side and a second or interior side 82. Exterior side 80 is exposed relative to opening 70 of housing 30, whereas interior side 82 defines a portion of port chamber 44. While FIG. 3 is described with regard to a fill port assembly 40, it will be understood the components described with regard to FIG. 3 can be readily applied or adapted to the catheter access port assembly 40′.

Although not depicted in FIG. 3, infusion device 12 may also include components such as safety valves, flow regulators and other components that may enhance the implanted infusion device's operation. Such components include those described in, for example, U.S. Pat. Nos. 6,203,523 and 6,048,328, both to Haller et al. both of which are incorporated herein by reference.

After infusion device 12 is implanted within patient, reservoir 32 can conveniently be accessed percutaneously to refill, flush or change the therapeutic substance stored within reservoir 32. For example, reservoir 32 may be refilled every few weeks or every few months, depending upon the capacity of reservoir 32 and the desirable agent delivery rate for a patient.

The disclosed needle system assists a HCP in obtaining the accurate needle placement within the appropriate chamber or other portion of an implanted medical device, and not in the patient's tissue, before fluid is dispensed.

Needle 20 may be any instrument that may be used to pierce through a patient's tissue to enter septum 42 and deliver a therapeutic substance into device 12. After needle 20 passes through septum 42, a therapeutic substance may be released from syringe 18 through the distal end of needle 20 into reservoir 32. Percutaneous direct fluid delivery to a patient may also be accomplished by introducing needle 20 or another medical instrument through catheter access port assembly 40′. Catheter access port assembly 40′ provides a sealed structure through which fluid may directly flow to catheter 34, thereby effectively bypassing reservoir 32.

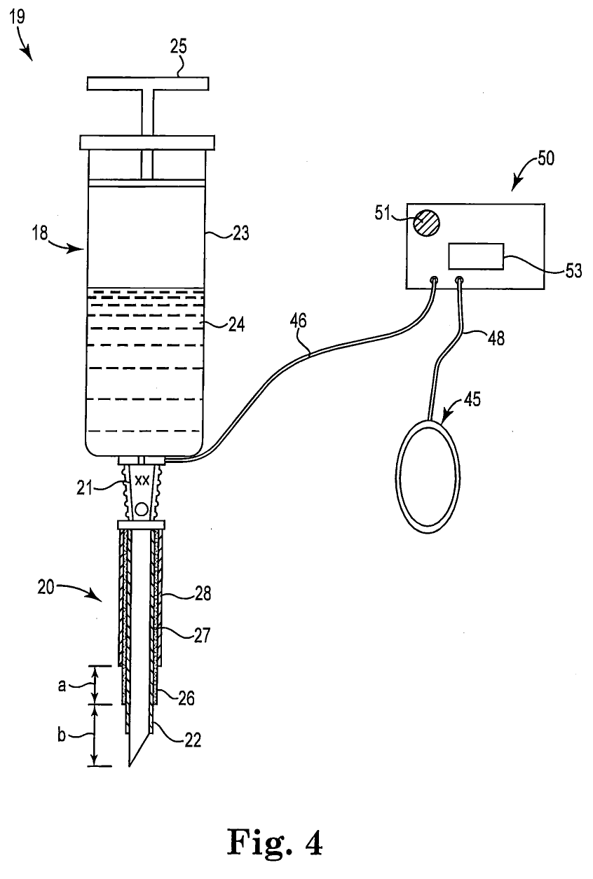

An embodiment of a needle device 19, shown in FIG. 4 includes needle 20, connected to a syringe 18 that contains a therapeutic substance 24 in fluid form. The syringe 18 includes a fluid-containing vessel or barrel 23 that receives and retains therapeutic substance 24. A plunger 25 may be inserted into syringe 18 to deliver therapeutic substance 24. The needle 20 is connected to the syringe 18 via a hub 21. Hub 21 may, for example, be a LUER connection or the like. Hub 21 may be formed from metal or polymer materials such as acrylonitrile butadiene styrene (ABS), polystyrene, polyvinyl chloride, polysulfone or other suitable material.

Needle 20 may be, for example, a conventional hypodermic or infusion needle, or another instrument that may be capable of piercing through a patient's tissue and entering an implanted infusion device 12, and delivering therapeutic substance into reservoir 32. Needle 20 desirably is made from a conductive material such as a metal or a metallic alloy. In other embodiments a non-conducting needle may be made conductive by coating with suitable conductive material such as a metal, alloy, carbon black, conductive polymer or other conductive material. Exemplary conductive coatings include thin film conductive traces, conductive foils, and conductive deposits formed using thin-film deposition techniques such as vapor deposition, metal plating, PVD sputter deposition and the like. Suitable conductive materials include, for example, aluminum, copper, gold, silver, nickel, iron, stainless steel, nitinol, composite conductive polymers and the like. Needle 20 may be removably coupled to hub 21 and may be designed for either single use or reuse. Exemplary needles include non-coring Huber needles, standard 22 gauge; angled non-coring Huber type needle, 22 gauge or 20-25 gauge; straight non-coring Huber type needle, 20-25 gauge; angled and straight safety non-coring Huber type needles, 20-25 gauge; non-coring infusion sets for I.V. port access such as Bard Wing Infusion set by Bard Medical or the like.

Referring to FIG. 4, needle 20, which may be an electrically conducting needle, may be covered by insulating layer 22 along the entire needle length. Insulating layer 22 is then covered by a conductive layer 27. Conductive layer 27 extends to the bottom of a over insulating layer 22. A second insulating layer 28, which covers the top of conductive layer 27, extends toward hub 21 and stops above a, creating electrode 26. In other embodiments, the needle may be a non-conducting needle that is made conductive at designated sections on the needle (as described below). These conductive sections serve as electrodes.

The position of electrode 26 may, as shown in FIG. 4, be at a distance b from the distal end of needle, i.e. the needle tip and have a length a such that when the tip of needle 20 is fully inserted into reservoir 32, electrode 26 is enveloped by septum 42. It should be understood that the position and length of electrode 26 is dictated by the device used. The length a of electrode 26 may be, for example, less than the depth of a septum. This way, electrode 26, when fully inserted in reservoir 32 will be completely enveloped by septum 42 and no electrode portion will be exposed in the tissue or in the reservoir. For example, electrode 26 may be positioned about 0.2 to about 0.3 inches from the distal tip of needle 20 with the length of the electrode about 0.05 to about 0.15 inches.

Depending on the electrical conducting characteristic of septum 42, for example, if non-conducting, septum 42 insulates electrode 26 and blocks current to a return electrode resulting in higher resistance or higher impedance compared to the resistance or impedance when electrode 26 is not enveloped by septum 42. Exemplary needle insulating materials include titanium dioxide, polytetrafluoroethylene (PTFE), PARYLENE™ polymers, AMC141-18 polymers from Advanced Materials Coatings, nylon and other polyamides and the like.

The needle electrode 26 may be electrically coupled to wire 46 via hub 21, which in turn is electrically coupled to detector 50. In some embodiments, a return electrode or ground pad 45 is separately provided and electrically coupled to the detector 50 by cables or wires 48. The return electrode 45 may be a surface electrode, for example, a standard ECG pads, such as the Conmed Suretrace ECG electrode. The surface electrode such as the ECG pad may be placed at a desirable position on a patient and the surface electrode returns the current or other electrical characteristic distributed from the needle electrode to the detector through the cables or wires to complete the electrical circuit. In other embodiments, a standard electrically conductive needle may be separately inserted into a patient's skin a small distance from the needle 20 to serve as a return electrode. In still other embodiments, needle 20 may include an additional electrode on the needle 20 which may serve as a return electrode.

Detector 50 may include a signal adjustment 51 and a display 53. Signal adjustment 51 may regulate the applied voltage, applied frequency, allowable current or other signal between wires 46 and 48. Detector 50 may receive multiple electrical signals and may compute, display or store information based on such signals. Detector 50 may also provide an audible, tactile, visual or other indication or cue to the user to show needle status or location within the body tissue or within various components of an implanted infusion device, such as a septum. If desired, the detected electrical characteristics may be outputted wireless from detector 50.

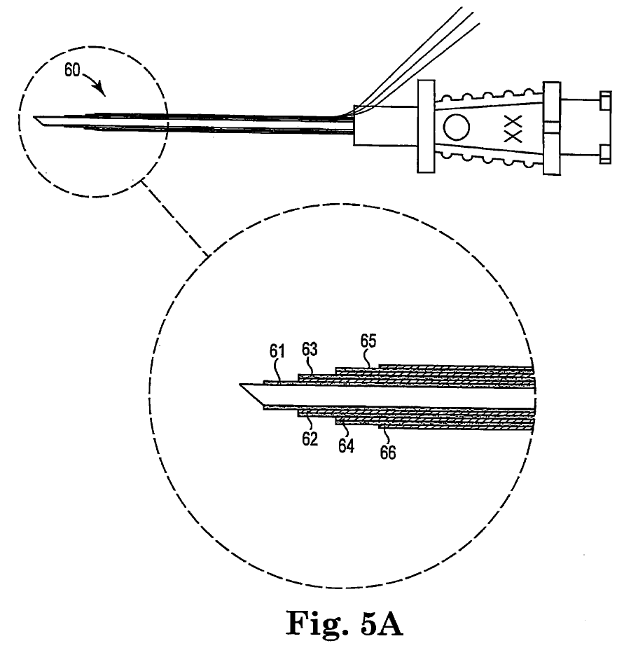

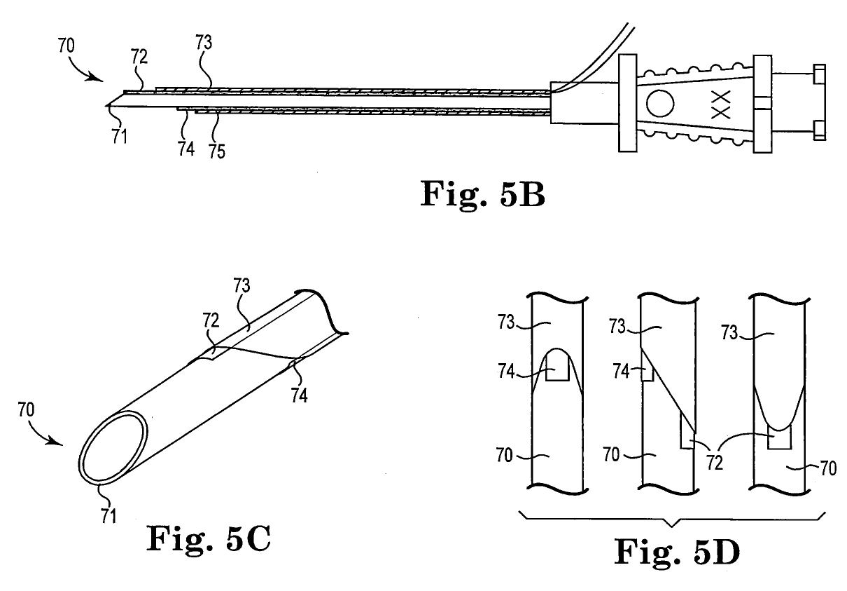

As shown in FIG. 5A, multiple needle electrodes may be provided on a non-conducting needle 60 in a multilayered configuration. Conductive layers 61, 63, & 65 may be deposited in an alternating arrangement with insulating layers 62, 64 & 66. The insulating layers 62, 64 & 66 may be disposed on each conductive layer 61, 63, & 65 such that a designated section or sections of the underlying conductive layer are partially or fully exposed. The partially or fully exposed sections serve as electrodes 61, 63, & 65. It will be understood that a variety of other methods for forming a needle with multiple electrodes may be used and that the needle used to form the multiple electrodes may be a conductive or non-conductive needle.

FIG. 5B shows a further embodiment with multiple electrodes arranged at different depths along the needle. Such a multiple electrode needle 70 includes a non-conducting needle 71 that is made conductive by providing conductive strips or traces 72, 74 along the needle sides as opposed to conductive layers along the entire needle circumference. Conductive strips 72, 74 are covered by insulating layers 73, 75. Uninsulated sections 72, 74 act as electrodes and are electrically coupled to detector 50. In some embodiments, where the needle is a conductive needle, the needle tip may also serve as an electrode. It will be understood that additional electrodes at varying depths may be created as described here.

FIGS. 5C & D show yet another embodiment where electrodes 72, 74 are provided as strips 72, 74 and at different depths and where a single insulating layer 73 is disposed on each conductive strip 72, 74 such that designated section or sections of the underlying conductive strips 72, 74 are partially or fully exposed to form the electrodes.

When a multiple electrode needle is used, the needle system may measure changes in electrical characteristics such as changes in electrical resistance (when direct current is used), impedance (when alternating current is used), current, voltage, frequency, or signal strength between multiple needle electrodes or between each electrode and a separate return electrode. While FIGS. 5A-D depict two or three electrodes, depending on the required needle placement resolution, additional electrodes may be included on the needle with or without a separately provided return electrode.

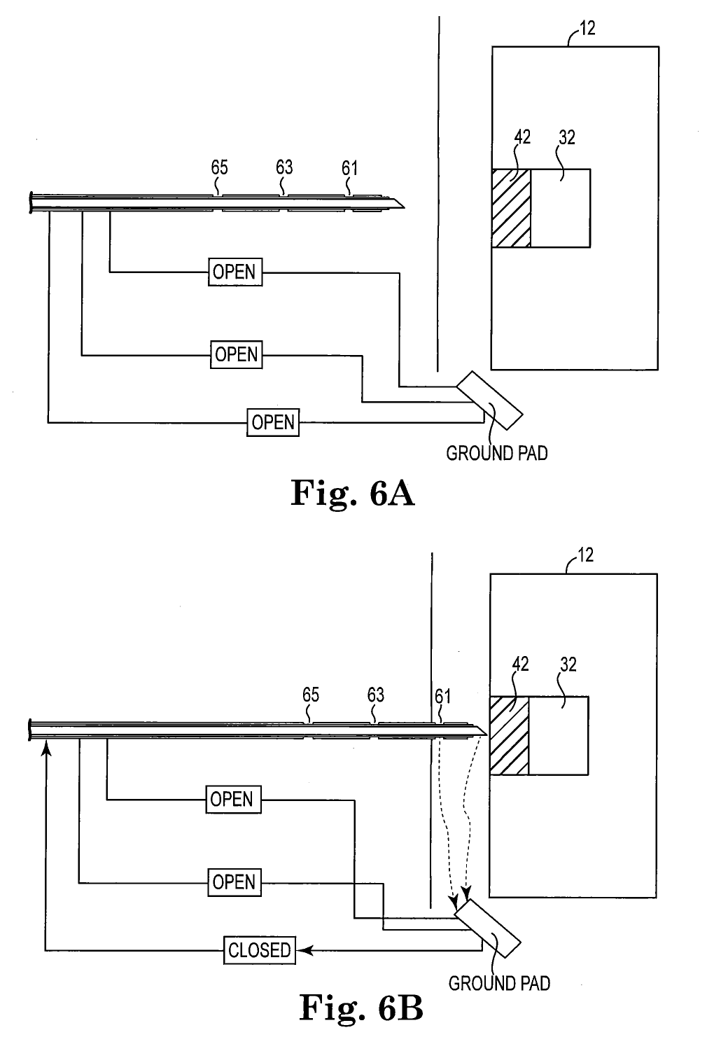

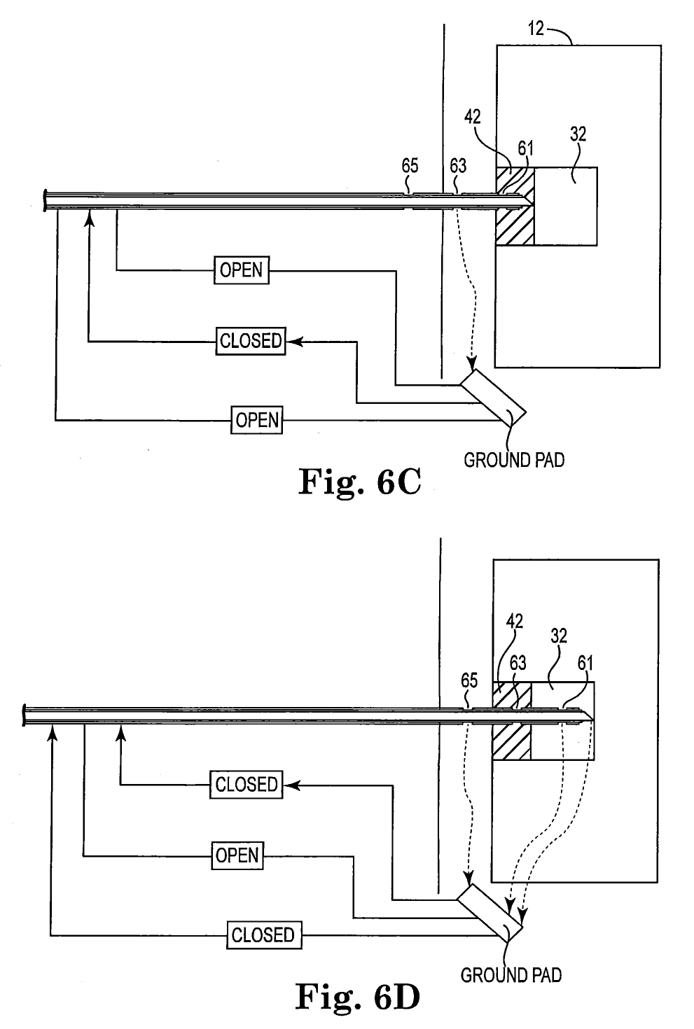

FIGS. 6A-D show various needle insertion stages and the expected condition of various electrical circuits as the needle is inserted into an implanted infusion device 12. In some embodiments, needle position may be determined with reasonable precision by simply monitoring the available circuits to determine if they are open or closed. It should be understood that by “open circuit” is meant an intact circuit but for the presence of the non-conducting material that resists or impedes current flow and not a circuit with a physical, actual gap or broken connection (disconnected wires). An open circuit for the purposes of this disclosure would result in higher impedance, whereas a circuit that has a gap or disconnected wires would result in infinite or unmeasurable impedance.

Greater precision may be obtained by monitoring a factor such as resistance (when direct current is employed) or impedance (when alternating current is employed), as doing so can indicate the extent to which any particular electrode has advanced along the needle insertion path through a zone in which the surrounding material (e.g. air, skin, percutaneous tissue, septum or reservoir fluid) changes to another material. Characteristic resistance values for subcutaneous tissue, the electrode(s) and their insulating sections, the septum, and the therapeutic substance may be measured to help determine needle position under various conditions. In one desirable embodiment, the highest resistance is obtained when the needle is correctly located in the septum. This can help the user of the needle, typically a HCP, detect when the distal end of needle 60 properly projects through the septum 42. As shown in FIG. 6D, for example, multiple electrodes may be spaced along the needle length such that when the distal tip of needle 60 reaches the bottom of reservoir 32 and electrode 61 is also in reservoir 32, electrode 63 is enveloped by septum 42 and electrode 65 is in the patient's tissue.

Detector 50 may, for example, continuously indicate a high current flow, or low resistance or impedance value while the distal end of the needle is advanced through the patient's skin on its way into the reservoir 32 via septum 42 (see FIG. 6B). When the distal end of the needle and its electrode 61 enters into a non-conducting septum 42 (see FIG. 6C), the current flowing between electrode 61 and ground pad decreases while resistance or impedance increases. The increase indicates to the user that electrode 61 has entered septum 42. The user then may further advance the needle into device 12 until the indicated resistance or impedance decreases sufficiently to indicate that the needle is properly positioned within reservoir 32 to permit therapeutic substance delivery. It will be understood that impedance between electrodes 61 and 63, 61 and 65 or 63 and 65 may also be measured as opposed to between individual needle electrodes 61, 63 or 65 and the ground pad.

It should be understood that while the needle has been described as conductive and as passing through a non-conductive septum of an implanted infusion device, the respective electrical properties of the needle and particular implanted infusion device component may be altered, e.g. reversed with respect to one another. For example, the needle may be uninsulated along all or most of its length, and may indicate its position by interacting with an electrically conductive implanted infusion device or component thereof.

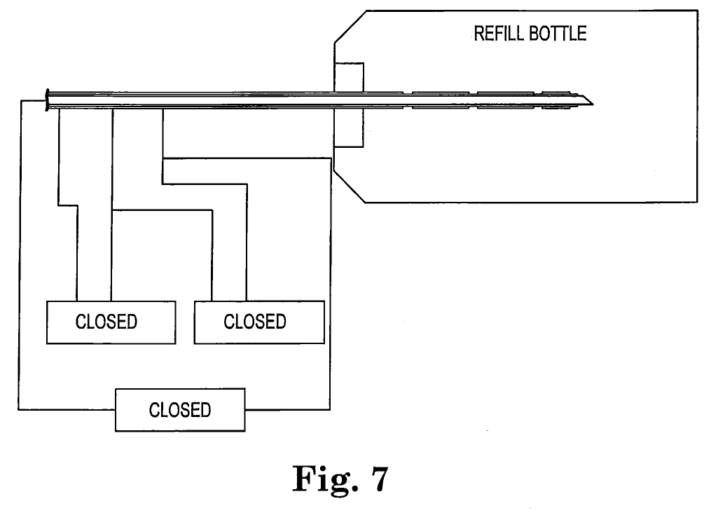

FIG. 7 shows a needle being inserted into a refill bottle. Needle placement into a refill bottle may serve as an initial system integrity check or for calibration of the needle system. For example, needle insertion until all the electrodes are immersed into a saline solution or the therapeutic substance maybe used as a check to confirm that all circuits are complete or that electrical characteristics such as voltage, resistance, impedance, current, frequency or signal strength values have expected or appropriate values in each circuit. If the system appears intact, the syringe can be filled and the needle used to deliver the syringe contents to a reservoir or catheter.

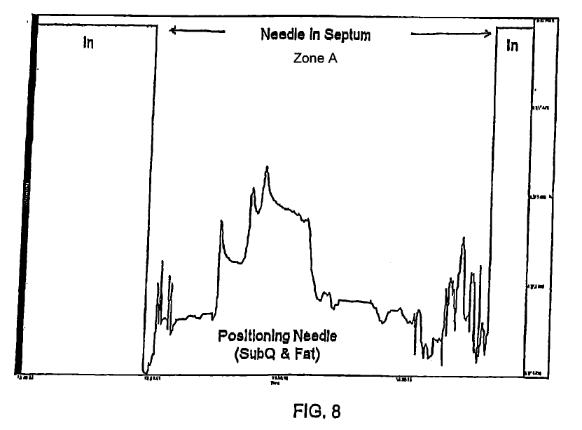

FIG. 8 shows an exemplary impedance profile associated with needle insertion through septum 42 into reservoir 32. The impedance profile shown in FIG. 8 was obtained by inserting a modified HUBER™ needle into the septum of SYNCHROMED™ II pump (Medtronic, Inc.) placed subcutaneously (SubQ) in sheep. The needle modification involved coating the entire needle length with an insulating material (PARYLENE™ polymer) except for a 0.1″ wide band beginning 0.2″ from the distal end of the needle to provide a single electrode. Proper insertion depth is indicated by the zone A in FIG. 8.

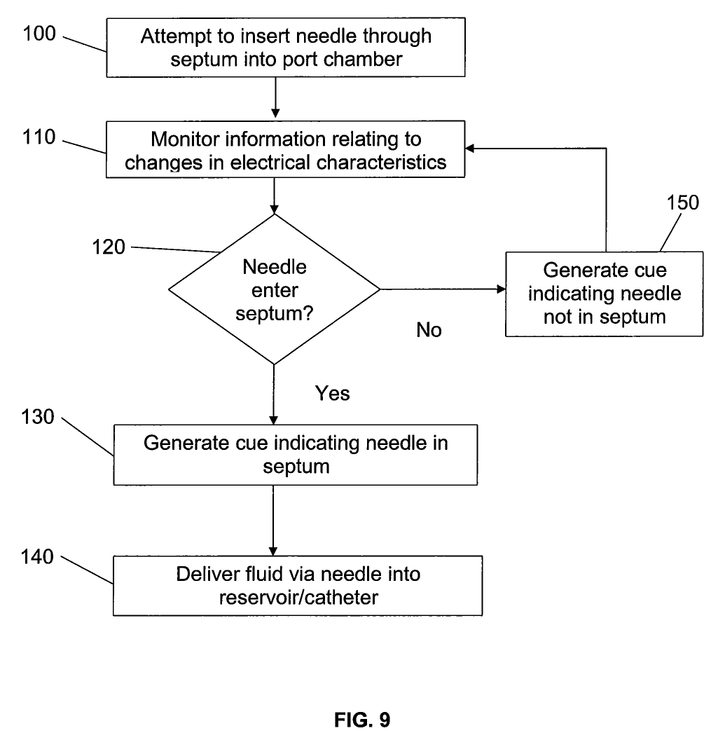

FIG. 9 shows a flow diagram illustrating a method for monitoring and detecting needle insertion into a septum. The method includes inserting a needle into a patient in an attempt to access an implanted infusion device (100) and monitoring changes in electrical characteristics (110). A determination may then be made as to whether the monitored information is indicative of needle insertion through a septum (120). If the monitored information is indicative of proper needle insertion into the reservoir or other component of the infusion device a cue may be generated (130) to alert the user of successful needle placement. The cue may take any form such as a visual, audible or tactile cue. The user may then proceed to deliver fluid into the reservoir or catheter via the needle (140). If information monitored is indicative of incorrect or insufficient needle insertion, no cue or a different cue may be generated (150), allowing the user to again attempt to insert the needle or reposition the needle (100) before fluid delivery.

We claim:

1. Needle detection system for determining needle position with respect to an implanted infusion device comprising:
a hollow needle having a sharp distal end;
a needle electrode located at a first distance along the needle from the distal end;
a second electrode; and
a detector electrically coupled to the first and second electrode, the detector providing needle insertion position information when the needle is inserted in an implanted medical device.

2. The system of claim 1, wherein the needle includes two or more electrodes.

3. The system of claim 1, wherein the second electrode is separate from the needle.

4. The system of claim 1, wherein the second electrode is an ECG pad.

5. The system of claim 1, wherein the second electrode is on the needle.

6. The system for claim 1, wherein the system visually, audibly or tactilely communicates needle position information.

7. The system of claim 1, wherein the detector evaluates changes in electrical characteristics.

8. The system of claim 1, wherein the detector evaluates changes in impedance.

9. The system of claim 1, wherein the detector evaluates an increase in impedance.

10. The system of claim 1, wherein the detector provides information that indicates needle insertion position with respect to a septum of the implanted infusion device.

11. The system of claim 10, wherein the detector provides information that indicates needle is inserted into a septum by an increase in impedance.

12. A position-indicating needle comprising:
a hollow needle having a sharp distal end; and
a needle electrode located at a first distance along the needle from a distal end; and
a second electrode.

13. The needle of claim 12, wherein the needle includes two or more electrodes.

14. The needle of claim 12, wherein the second electrode is separate from the needle.

15. The needle of claim 12, wherein the second electrode is an ECG pad.

16. The needle of claim 12, wherein the second electrode is on the needle.

17. The needle of claim 12, wherein the second electrode is the needle tip.

18. A method for detecting needle position with respect to an implanted infusion device, the method comprising:
sensing changes in electrical characteristics as the needle enters into an implanted infusion device; and
determining whether the sensed changes in electrical characteristics are indicative of needle entry into the implanted infusion device.

19. The method of claim 18, comprising sensing an impedance change.

20. The method of claim 18, comprising determining needle entry through a septum of the implanted device.

21. The method of claim 18, comprising determining needle entry through a septum of the implanted device by an increase in impedance.

22. The method of claim 18, further comprising generating an audible, tactile or visual cue indicating needle position.
